(19) United States
(12) Patent Application Publication
(10) Pub. No.: US 2026/0224263 A1
(43) Pub. Date: Aug. 6, 2026

(54) SHEATH DESIGN FOR MEDICAL DEVICE

(71) Applicant: Affera, Inc., Newton, MA (US)

(72) Inventor: Chak M. Leung, Palo Alto, CA (US)

(21) Appl. No.: 19/460,499

(22) Filed: Jan. 27, 2026

Related U.S. Application Data
(60) Provisional application No. 63/752,236, filed on Jan. 31, 2025.

(51) Int. Cl. A61B 18/00

(52) U.S. Cl. CPC A61B 18/00; A61B 2018/00184; A61B 2018/00214; A61B 2018/00577; A61B 2018/00821

(57) ABSTRACT

A catheter includes an elongate body defining a longitudinal axis. The elongate body includes an expandable structure coupled to a distal end of the elongate body, and a plurality of outer radial elements that define an outer profile of the expandable structure. Each one of the outer radial elements is radially disposed about the longitudinal axis in a symmetrical configuration. The catheter also includes a sheath movable along the elongate body between a first configuration where the expandable structure is located outside the sheath, and a second configuration where the expandable structure is located within the sheath. The sheath includes a distal face, and the outer radial elements engage each other in an offset manner such that less than all of the outer radial elements engage the distal face of the sheath at a given moment in time as the sheath receives the expandable structure.

### CROSS-REFERENCE TO RELATED APPLICATIONS

This application claims priority to U.S. Provisional Patent Application 63/752,236, filed on January 31, 2025, the entire contents of which are incorporated herein by reference.

### BACKGROUND

As an alternative to open-heart surgery, many medical procedures are performed using minimally invasive surgical techniques, where one or more slender implements are inserted through one or more small incisions into a patient’s body. Such procedures may involve the use of catheters or probes having multiple sensors, electrodes, or other measurement and treatment components to treat the diseased area of the heart, vasculature, or other tissue. In some designs of catheters, the sensors or electrodes are expandable elements that are expanded within the patient’s body and retracted again before removal from the patient’s body. When the sensors or electrodes are expanded and contracted, friction is generated between the sensors/electrodes and the implement. Therefore, repeat medical procedures utilizing the same implement can cause wear and tear to the implement housing because the sensors/electrodes are repeatedly expanded/retracted within the implement.

### SUMMARY

One example provides, among other things, a catheter that includes an elongate body defining a longitudinal axis. The elongate body includes a proximal end and a distal end, an expandable structure including a first end coupled to the distal end of the elongate body and a second end opposite the first end, and a plurality of outer radial elements that define an outer profile of the expandable structure. Each one of the plurality of outer radial elements is radially disposed about the longitudinal axis in a symmetrical configuration. The catheter also includes a sheath movable along the elongate body between a first configuration where the expandable structure is located outside the sheath, and a second configuration where the expandable structure is located within the sheath. The sheath includes a distal face, and the outer radial elements are configured to engage each other in an offset manner such that less than all of the plurality of outer radial elements engage the distal face of the sheath at a given moment in time as the sheath receives the expandable structure.

Another example provides, among other things, a sheath for use with a catheter that includes an elongate body defining a longitudinal axis, and an expandable structure located on the elongate body. The expandable structure includes a plurality of sensors that are each radially disposed about the longitudinal axis in a symmetrical configuration. The sheath includes an opening, and a transition section adjacent the opening configured to facilitate movement of the sheath along the elongate body between a first configuration where the expandable structure is located outside the sheath, and a second configuration where the expandable structure is located within the sheath. The transition section biases the plurality of sensors from the symmetrical configuration about the longitudinal axis to an asymmetrical configuration about the longitudinal axis as the sheath is moved from the first configuration to the second configuration.

Other examples, embodiments, aspects, and features are described below.

### BRIEF DESCRIPTION OF THE SEVERAL VIEWS OF THE DRAWINGS

The accompanying figures, where like reference numerals refer to identical or functionally similar elements throughout the separate views, together with the description below, are incorporated in and form part of the specification, and serve to further illustrate embodiments, examples, aspects, and features that include the claimed subject matter, and explain various principles and advantages.

FIG. 1 is a perspective view of an example of a medical device utilizing a sheath in accordance with some aspects.

FIG. 2 is a zoomed-in side view of the medical device of FIG. 1.

FIG. 3 is a schematic view of a sheath and an assembly in accordance with some aspects.

FIG. 4 is a schematic view of a sheath and an assembly in accordance with other aspects.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments, examples, aspects, and features.

The apparatus and method components have been represented where appropriate by conventional symbols in the drawings, showing only those specific details that are pertinent to understanding the embodiments, examples, aspects, and features described and illustrated so as not to obscure the disclosure with details that will be readily apparent to those of ordinary skill in the art having the benefit of the description herein.

### DETAILED DESCRIPTION

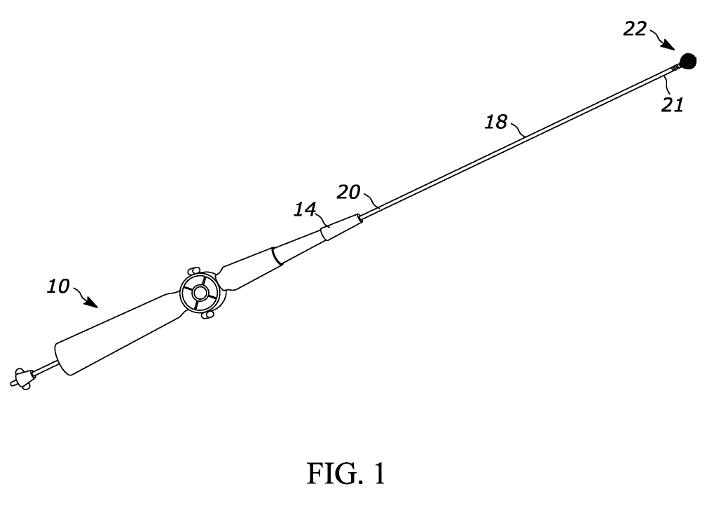

FIG. 1 illustrates a medical device 10 that may be coupled to a generation unit (not shown) or operating console. The medical device 10 may generally include one or more diagnostic or treatment regions for energetic, therapeutic, and/or investigatory interaction with a patient. For example, the treatment region(s) may be configured to deliver ablation energy (e.g., cryogenic therapy, radiofrequency energy, and/or pulsed field ablation energy) to cardiac tissue of the patient once the tissue is in proximity to the treatment region(s).

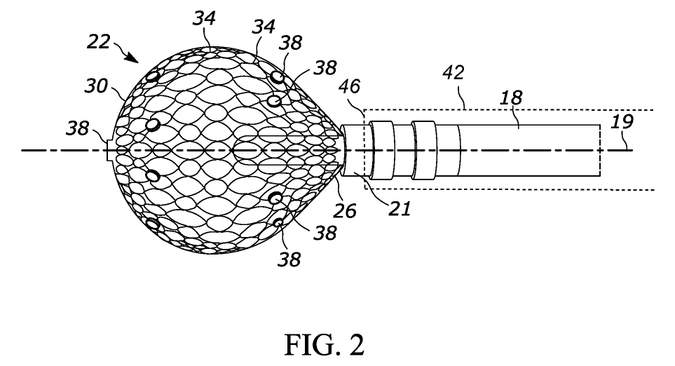

In the example shown in FIGS. 1 and 2, the medical device 10 (e.g., a catheter) includes an insertion tool 14 (e.g., an intravascular introducer) for inserting an elongate body 18 within a patient’s vasculature and/or proximate to a tissue region for diagnosis, treatment, and/or mapping. The elongate body 18 defines a longitudinal axis 19 (FIG. 2) and includes a proximal end 20 adjacent the insertion tool 14 and a distal end 21 opposite the proximal end 20. The medical device 10 also includes an assembly 22 extending longitudinally from a proximal first end 26 to a distal second end 30, where the assembly 22 is configured for diagnosis, treatment, and/or mapping. The assembly 22 is located on the distal end 21 of the elongate body 18.

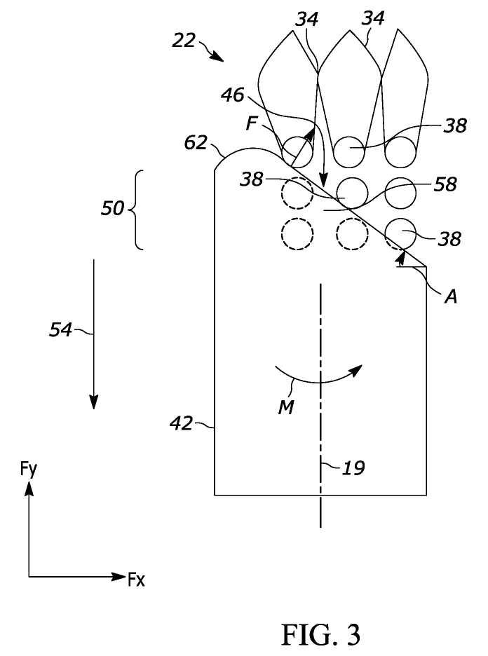

In the examples shown in FIGS. 2 and 3, the medical device 10 includes a sheath 42 surrounding the elongate body 18 and movable relative to the elongate body 18. The sheath 42 includes an opening 46 to a lumen, from which the assembly 22 is selectively deployed and received (e.g., received following diagnosis or treatment in order to withdraw the medical device 10 from a patient). In some examples, the assembly 22 is configured to expand into a larger profile once deployed, and collapse into a stowed configuration for navigation through the patient vasculature. Withdrawing the assembly 22 into the sheath 42 to be received applies radial force inward upon the assembly 22, where this radial force may cause undesired mechanical stress to the medical device 10. For example, outer radial elements 38 that define an outer profile of the assembly that are symmetrically radially disposed about the longitudinal axis 19 on the lattice structure 34 may undergo undesired mechanical stress during withdrawal of the assembly 22 into the sheath 42.

Aspects of this disclosure relate to the assembly 22 being physically configured to engage the sheath 42 in an offset manner such that less than all of the plurality of outer radial elements 38 engage the distal face of the sheath 42 at a given moment in time as the sheath 42 receives the expandable structure of the assembly 22. For example, aspects of the disclosure relate to a relatively reduced number of radial elements 38 of the assembly 22 introducing friction with the sheath 42 at a single moment. Comparatively, in some conventional medical devices, there may be numerous outer elements on an example assembly on a single longitudinal plane, where each of these outer elements engage the distal face of a respective sheath at substantially the same moment in time when the respective sheath is receives the respective conventional assembly. Conversely, aspects of this disclosure relate to outer elements on an assembly 22 being longitudinally offset so that they do not engage the sheath 42 all at once. Additionally, or alternatively, aspects of this disclosure relate to a distal surface of the sheath 42 being angled (e.g., angled in a matter similar to what is depicted in FIG. 3) such that outer elements on an assembly 22 are not engaging the sheath 42 all at once (e.g., whether or not the outer elements of an assembly 22 are longitudinally offset or whether there are numerous elements that share a single longitudinal plane).

The assembly 22 includes a first end 26 coupled to the distal end 21 of the elongate body 18 and includes a lattice structure 34 extending from the first end 26 to the second end 30 of the assembly 22 that is radially disposed about the longitudinal axis 19. In some examples, the lattice structure 34 functions as an ablation electrode that ablates tissue via ablation energy generated by the generation unit or operating console. As noted above, the assembly 22 further includes a plurality of outer radial elements 38 supported on the lattice structure 34 that are each symmetrically radially disposed about the longitudinal axis 19 on the lattice structure 34. The outer radial elements may define an outer radial profile of the assembly 22 at various planes perpendicular to the longitudinal axis. The outer radial elements 38 may be configured to assist or enable the diagnosis, mapping, and/or treatment functionality of the medical device 10. For example, the outer radial elements 38 may be sensors or electrodes. In other examples, the outer radial elements 38 may be structural elements that are for purposes of maintaining structural integrity as the assembly 22 is deploying, or otherwise being maneuvered. For purposes of discussion, outer radial elements 38 are referred to as sensors predominantly herein. Each of the plurality of outer radial elements 38 are structurally supported on the lattice structure 34 via rivets. In some examples, each of the plurality of outer radial elements 38 are thermocouples for providing real-time tissue proximity information and tissue temperature feedback to the user. In other examples, the plurality of outer radial elements 38 can be arranged in one or more rows axially spaced from each other along the longitudinal axis 19. In other examples, the plurality of outer radial elements 38 are sensing or mapping electrodes for obtaining a baseline electrophysiological map of electrical activity in selected tissue of the treatment regions(s). In yet other examples, the plurality of outer radial elements 38 are nine total sensors arranged in two spaced apart rows in addition to a single sensor placed at the second end 30 of the assembly 22.

In one example, the sheath 42 and assembly 22 are selectively movable relative to each other along the elongate body 18 along the longitudinal axis 19 between a first configuration (FIG. 2) where the assembly 22 is located outside the sheath 42 and fully expanded (e.g., expanded to form a substantially spherical lattice structure), and a second configuration (not shown) where the assembly 22 is completely retracted and housed within the sheath 42. The sheath 42 and assembly 22 are configured to be in the second configuration when the elongate body 18 is inserted within and navigated through a patient’s vasculature and moved to the first configuration when the distal end 21 of the elongate body 18 is proximate to the tissue region for diagnosis, treatment, and/or mapping. In other examples, the assembly 22 can form other structural shapes when fully expanded in the first configuration. For example, the assembly 22 can be expanded into a loop, a flower petal configuration, or the like.

In FIG. 3, the sheath 42 is illustrated as having a transition section 50 located adjacent the opening 46 of the sheath 42. The transition section 50 has a particular geometry for facilitating the expansion and retraction of the assembly 22 within the sheath 42 along an insertion direction 54 parallel to the longitudinal axis 19. This is in contrast to a conventional medical device with like components and features of the medical device 10, which has relatively less of a transition section (in some examples, a conventional medical device has functionally no transition section). As a result of there being no transition section, as a respective assembly is retracted within a sheath along a proximal insertion direction that is parallel to a longitudinal axis defined by the sheath, a plurality of outer radial elements (e.g., sensors, or electrodes, or some other structural element) may all contact an outer circumference of an opening of the sheath at the same time, thereby creating a sudden jump in frictional contact. Specifically, the jump in frictional contact might be between the assembly of the conventional medical device and the sheath. Depending on the specific geometry of the conventional medical device, the jump in frictional force might be accompanied by a large tension force between the plurality of outer radial elements contacting the opening of the sheath and the structure on which the outer radial elements are attached. Over time, repeated frictional contact can damage the sheath and the plurality of outer radial elements.

One cause of this frictional contact is the symmetrical arrangement of the plurality of outer radial elements about the longitudinal axis. The symmetry causes the plurality of radial elements to contact the sheath at the same time (e.g., at approximately a 45-degree angle relative to the longitudinal axis) when the assembly is folding within an opening of the sheath. To reduce this undesirable frictional contact, aspects of this disclosure relate to the sheath 42 utilizing the transition section 50.

Returning to FIG. 3, in some instances, the transition section 50 includes a sloped portion 58 defining the opening 46, and a shoulder portion 62 rounded to reduce damage to the tissue region for treatment. The sloped portion 58 is shaped to help prevent the plurality of outer radial elements 38 from all contacting an outer circumference of the opening 46 of the sheath 42 at the same time when each of the outer radial elements 38 are in the symmetrical configuration. In one example, the sloped portion 58 acts to displace each of the plurality of outer radial elements 38 from the symmetrical configuration about the longitudinal axis 19 to an asymmetric configuration about the longitudinal axis 19, where each of the plurality of outer radial elements 38 are staggered to sequentially contact the outer circumference of the opening 46 as the assembly 22 enters the sheath 42 along the insertion axis 58. To promote sequential contact of the plurality of outer radial elements 38 within the sheath 42, an angle A of the sloped portion 58 is formed such that a rotational period of the sloped portion 58 is offset from a rotational period of each of the plurality of outer radial elements 38 (e.g., offset by 90 degrees). In this example, contact between each one of the plurality of outer radial elements 38 and the sloped portion 58 of the sheath 42 imparts a biasing force F onto each one of the plurality of outer radial elements 38 that is normal to the sloped portion 58. The biasing force F can be separated into a lateral force component Fx normal to the longitudinal axis 19 and an axial force component Fy co-axial with the longitudinal axis 19.

As the assembly 22 is being retracted and folded within the sheath 42 along the insertion axis 54, the lateral force Fx biases each one of the plurality of outer radial elements 38 and corresponding individual elements of the lattice structure 34 laterally perpendicular to the longitudinal axis 19. The lateral force Fx causes each of the plurality of outer radial elements 38 and corresponding individual elements of the lattice structure 34 to rotate locally in a rotational direction M about the longitudinal axis 19. This rotation causes a rolling or sliding frictional contact between plurality of outer radial elements 38 and the sloped portion 58, thereby enabling each one of the plurality of outer radial elements 38 to traverse down the sloped portion 58, over the outer circumference of the opening 46, and into the sheath 42 all while rotating in the rotational direction M about the longitudinal axis 19. This action allows the assembly 22 to laterally compress to fold neatly within the sheath 42, while maintaining the constant axial force Fy.

With continued reference to the example shown in FIG. 3, the lattice structure 34 is laterally flexible and axially stiff along the longitudinal axis 19 due to the lattice structure of the assembly 22. In some instances, the transition section 50 is shaped to bias each of the plurality of outer radial elements 38 from the symmetrical configuration to the asymmetrical configuration during retraction within the sheath 42. As a consequence, once the assembly 22 is housed within the sheath 42, the plurality of outer radial elements 38 move back to the symmetrical configuration due to a spring back force. In some instances, the spring back force is created due to inherent elastic properties of the lattice structure 34 returning to its natural shape when the assembly 22 is in the first configuration outside the sheath 42.

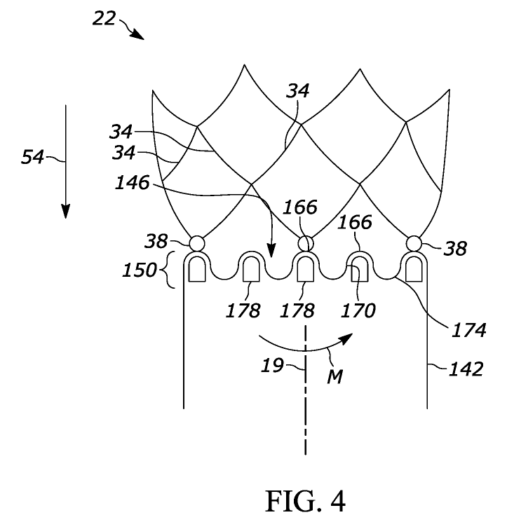

FIG. 4 illustrates an alternative example of a sheath 142 for use with the medical device 10. Like components and features of the sheath 42 are identified with reference numerals that are incremented by 100. In this example, the sheath 142 includes an opening 146 to a lumen, where the assembly 22 is selectively received. In some instances, the sheath 142 and assembly 22 are selectively movable along the elongate body 18 (FIG. 2) along the longitudinal axis 19 between a first configuration where the assembly 22 is located outside the sheath 142 and fully expanded to form a substantially spherical lattice structure, and a second configuration where the assembly 22 is retracted and housed within the sheath 142. The sheath 142 also includes a transition section 150 located adjacent the opening 146 of the sheath 142. In some instances, the transition section 150 has a particular geometry for facilitating the expansion and retraction of the assembly 22 within the sheath 142 along the insertion direction 54 parallel to the longitudinal axis 19.

In an example shown in FIG. 4, the transition section 150 includes a distal face of the sheath 142 defining a plurality of sloped protrusions 166 radially disposed about the longitudinal axis 19 that together form an outer circumference of the opening 146 of the sheath 142. Each of the plurality of sloped protrusions 166 includes an angled portion 170 that defines valleys 174 between successive protrusions 166, and a cut-out portion 178 formed as a through-hole extending through the thickness of the sheath 142. In other examples, each of the plurality of protrusions 166 do not include the cut-out portion 178. The plurality of protrusions 166 are formed to reduce the resistance between the sheath 142 and assembly 22 during insertion of the assembly 22 into the sheath 142 along the insertion axis 54. In some instances, the plurality of sloped protrusions 166 are shaped to help prevent the plurality of outer radial elements 38 from all contacting the outer circumference of the opening 146 of the sheath 142 at the same time when each of the outer radial elements 38 are in a symmetrical configuration about the longitudinal axis 19. In one instance, each of the plurality of protrusions 166 act to displace each of the plurality of outer radial elements 38 from the symmetrical configuration to an asymmetric configuration about the longitudinal axis 19, where the plurality of outer radial elements 38 are staggered (e.g., staggered axially relative to the protrusions 166) to sequentially contact the outer circumference of the opening 146 as the assembly 22 enters the sheath 142 along the insertion axis 54. In certain instances, the sequential contact is achieved by contouring the angled portion 170 of each of the plurality of sloped protrusions 166, or varying the number of the plurality of sloped protrusions 166 about the longitudinal axis 19 to offset the rotational period of the plurality of sloped protrusions 166 and the plurality of outer radial elements 38 by, for example, 90 degrees.

In some instances, as the assembly 22 is retracted and folded within the sheath 142 along the insertion axis 54, each of the plurality of outer radial elements 38 moves laterally perpendicular to the longitudinal axis 19. This lateral movement causes rolling or sliding frictional contact between each of the plurality of outer radial elements 38 and the sloped protrusions 166, causing each of the outer radial elements 38 to slide down the angled portion 170 of each of the respective sloped protrusions 166 in the rotational direction M about the longitudinal axis 19. By sliding down, the angled portions 170 of the respective plurality of sloped protrusions 166, each of the outer radial elements 38 locally deforms each of the respective sloped protrusions 166 radially outward from the longitudinal axis 19 to allow each of the outer radial elements 38 to pass over the outer circumference of the opening 146 and into the sheath 142.

With continued reference to FIG. 4, like the sheath 42, once the assembly 22 is housed within the sheath 142, the plurality of outer radial elements 38 move back to the symmetrical configuration due to the spring back force generated from the inherent elastic properties of the lattice structure of the lattice structure 34 returning to its natural shape.

By utilizing either of the sheath 42, 142 designs in a medical device, damage due to the high friction forces between the sensors/electrodes and the protective sheath is reduced.

In the foregoing specification, certain examples, embodiments, aspects, and features have been described. However, one of ordinary skill in the art appreciates that various modifications and changes can be made without departing from the scope of the claimed subject matter. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises …a,” “has …a,” “includes …a,” or “contains …a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art, and in one non-limiting example the term is defined to be within 10%, in another example within 5%, in another example within 1% and in another example within 0.5%. The term “coupled” as used herein is defined as connected, although not necessarily directly and not necessarily mechanically. A device or structure that is “configured” in a certain way is configured in at least that way but may also be configured in ways that are not listed.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A catheter comprising:
an elongate body defining a longitudinal axis, the elongate body including a proximal end and a distal end;
an expandable structure including
a first end coupled to the distal end of the elongate body and a second end opposite the first end,
a plurality of outer radial elements that define an outer radial profile of the expandable structure, each one of the plurality of outer radial elements radially disposed about the longitudinal axis in a symmetrical configuration, and
a sheath movable along the elongate body between a first configuration where the expandable structure is located outside the sheath, and a second configuration where the expandable structure is located within the sheath;
wherein a distal face of the sheath and the outer radial elements are configured to engage each other in an offset manner such that less than all of the plurality of outer radial elements engage the distal face of the sheath at a given moment in time as the sheath receives the expandable structure.

2. The catheter of claim 1, the catheter comprising a lattice structure radially disposed about the longitudinal axis and configured to extend from the first end to the second end of the expandable structure, wherein:
the outer radial elements are coupled to the lattice structure and radially disposed about the longitudinal axis in a symmetrical configuration, and
the sheath includes a transition section configured to facilitate movement of the sheath between the first configuration and the second configuration by biasing the plurality of outer radial elements from the symmetrical configuration about the longitudinal axis to an asymmetrical configuration about the longitudinal axis.

3. The catheter of claim 2, wherein the lattice structure functions as an ablation electrode.

4. The catheter of claim 2, wherein the plurality of outer radial elements are thermocouples.

5. The catheter of claim 1, wherein the distal face defines a plurality of sloped protrusions radially disposed about the longitudinal axis that define valleys therebetween.

6. The catheter of claim 5, wherein the plurality of sloped protrusions each include cut-out portions.

7. The catheter of claim 2, wherein the transition section includes a sloped portion.

8. The catheter of claim 7, wherein, when the sheath is moved between the first configuration to the second configuration, the sloped portion biases the plurality of outer radial elements in a lateral direction transverse the longitudinal axis to stagger contact between each of the plurality of outer radial elements and the sheath to form the asymmetrical configuration.

9. The catheter of claim 7, wherein, when the sheath is in the second configuration, the plurality of outer radial elements are biased back into the symmetrical configuration by a spring back force imparted by the lattice structure.

10. The catheter of claim 5, wherein the plurality of sloped protrusions are formed to create a rotational period that is 90 degrees offset from a rotational period of the plurality of outer radial elements.

11. A sheath for use with a catheter that includes an elongate body defining a longitudinal axis, and an expandable structure located on the elongate body, the expandable structure has a plurality of sensors that are each radially disposed about the longitudinal axis in a symmetrical configuration, the sheath comprising:
an opening; and
a transition section adjacent the opening configured to facilitate movement of the sheath along the elongate body between a first configuration where the expandable structure is located outside the sheath, and a second configuration where the expandable structure is located within the sheath;
wherein the transition section biases the plurality of sensors from the symmetrical configuration about the longitudinal axis to an asymmetrical configuration about the longitudinal axis as the sheath is moved from the first configuration to the second configuration.

12. The sheath of claim 11, wherein the elongate body further includes a proximal end and a distal end opposite the proximal end.

13. The sheath of claim 12, wherein the expandable structure further includes
a first end coupled to the distal end of the elongate body and a second end opposite the first end, and
a lattice structure radially disposed about the longitudinal axis and configured to extend from the first end to the second end of the expandable structure, and the plurality of sensors are coupled to the lattice structure.

14. The sheath of claim 11, wherein the transition section includes a sloped portion.

15. The sheath of claim 14, wherein, when the sheath is moved between the first configuration to the second configuration, the sloped portion biases the plurality of sensors in a lateral direction transverse the longitudinal axis to stagger contact between each of the plurality of sensors and the sheath to form the asymmetrical configuration.

16. The sheath of claim 13, wherein, when the sheath is in the second configuration, the plurality of sensors are biased back into the symmetrical configuration by a spring back force imparted by the lattice structure.

17. The sheath of claim 11, wherein the transition section includes a plurality of sloped protrusions radially disposed about the longitudinal axis that define valleys therebetween.

18. The sheath of claim 17, wherein the plurality of sloped protrusions each include cut-out portions.

19. The sheath of claim 17, wherein the plurality of sloped protrusions are formed to create a rotational period that is 90 degrees offset from a rotational period of the plurality of sensors.

20. The sheath of claim 11, wherein the plurality of sensors are thermocouples.
